(19) United States
(12) Patent Application Publication
(10) Pub. No.: US 2006/0084059 A1
(43) Pub. Date: Apr. 20, 2006

(54) Serum biomarkers in hepatocellular carcinoma

(76) Inventors: Tai-Tung Yip, Cupertino, CA (US); Terence C.W Poon, Castel Peak (CN); Philip Johnson, Oncology (GB); Victor F Yip, Union City, CA (US); Christine L Yip, Cupertino, CA (US); Anthony T.C Chan, The Chinese University (GB)

Correspondence Address: FOLEY AND LARDNER LLP;SUITE 500, 3000 K STREET NW, WASHINGTON, DC, 20007, US

(21) Appl. No.: 10/508,781

(22) PCT Filed: Apr. 7, 2003
(86) PCT No.: PCT/US03/10489

§ 371 Date: Sep. 19, 2005

Related U.S. Application Data
(60) Provisional application No. 60/370,239, filed on Apr. 8, 2002.

(51) Int. Cl. C12Q 1/68; G01N 33/574

(52) U.S. Cl. 435/6; 435/7.23

(57) ABSTRACT

Certain biomarkers and biomarker combinations are useful in a qualifying hepatocellular carcinoma status in a patient. A diagnostic methodology employing these biomarkers and combinations can distinguish between hepatocellular carcinoma and chronic liver disease, for example.

This application is based on U.S. provisional application No. 60/370,239, filed Apr. 8, 2002, and incorporated by reference herein.

### BACKGROUND OF THE INVENTION

The present invention relates generally to the field of serum biomarkers in hepatocellular carcinoma (HCC). More particularly, the invention relates to serum biomarkers that can distinguish HCC from other conditions, such as chronic liver disease and cirrhosis of the liver, respectively.

Globally, HCC is the eighth most common cancer, and the most common malignant tumor of males, with an incidence of 1 million new cases each year. It is responsible to approximately 1 million deaths each year, mainly in underdeveloped and developing countries. In the United States, the 5-year overall survival (1992-1996) rate is 5%. El-Serag et al., Hepatology 33:62-65 (2001). Liver dysfunction related to viral infection, e.g., from hepatitis B or C, alcoholic liver damage and alfatoxin B exposure, generally lead to malignant transformation. Indeed, 80% of HCC worldwide is etiologically associated with HBV, and HBV is estimated to account for one in four cases of HCC among non-Asians in the United States. There is no standard therapy and the prognosis is poor.

The conventional biomarker for HCC is alpha-fetoproteins (AFP). However, patients with chronic liver disease also have elevated serum levels of AFP. Since HCC typically arises in patients with coexisting chronic liver disease, AFP level alone is a poor biomarker, and has a cancer predictive value only in the 40% range. Quantitative analysis of isoforms of AFP can improve the diagnostic value to 75%, but is very time consuming, and labor intensive. In addition, about 20% of HCC patients have very low AFP levels, less than 20 ng/ml. Both the p53 protein and various aldehyde dehydrogenase isozymes have been tested as potential markers, however, none of these have a predictive value that is even as high as AFP.

Biopsy can be used to diagnose HCC, but it is an invasive procedure and, therefore, less than desirable. Other diagnostic methods for HCC include ultrasound and computed tomography (CT) scan. Only 25-28% of HCC nodules that are smaller than 2 cm can be detected by ultrasonography and CT scan during arterial portography.

It would be highly desirable to have a biomarker or combination of biomarkers capable not only of identifying HCC but also of distinguishing it from chronic liver disease (CLD), among other conditions. The literature on HCC diagnosis has not disclosed heretofore such a biomarker or combination of biomarkers, however.

### SUMMARY OF THE INVENTION

In accordance with the present invention, biomarkers and combinations of biomarkers are used to identify HCC. The method successfully distinguishes between HCC and CLD. In one embodiment, a method for qualifying hepatocellular carcinoma status in a subject comprises analyzing a biological sample from a subject for a diagnostic level of a protein selected from either a first group consisting of

(A) I-M1, I-M2, I-M3, I-M4, I-M5, I-M6, I-M7, I-M8, I-M9, I-M10, I-M11, I-M12, I-M13, I-M14, I-M15, I-M16, I-M17, I-M18, I-M19, I-M20, I-M21, I-M22, I-M23, I-M24, I-M25, I-M26, I-M27, I-M28, I-M29, I-M30, I-M31, I-M32, I-M33, I-M34, I-M35, I-M36, I-M37, I-M38, I-M39, I-M40, I-M41, I-M42, I-M43, I-M44, I-M45, I-M46, I-M47, I-M48, I-M49, I-M50, I-M51, I-M52, I-M53, I-M54, I-M55, I-M56, I-M57, I-M58, I-M59, I-M60, I-M61, I-M61, I-M62, I-M63, I-M64, I-M65, I-M66, I-M67, I-M68, I-M69, I-M70, I-M71, I-M72, I-M73, I-M74, I-M75, I-M76, I-M77, I-M79, I-M80, I-M81, I-M82, I-M83, I-M84, I-M85, I-M86, I-M87, I-M88, I-M89, I-M90, I-M91, I-M92, I-M93, I-M94, I-M95, I-M96, I-M97, I-M98, I-M99, I-M100

and/or a second group consisting of

(B) W-M1, W-M2, W-M3, W-M4, W-M5, W-M6, W-M7, W-M8, W-M9, W-M10, W-M11, W-M12, W-M13, W-M14, W-M15, W-M16, W-M17, W-M18, W-M19, W-M20, W-M21, W-M22, W-M23, W-M24, W-M25, W-M26, W-M27, W-M28, W-M29, W-M30, W-M31, W-M32, W-M33, W-M34, W-M35, W-M36, W-M37, W-M38, W-M39, W-M40, W-M41, W-M42, W-M43, W-M44, W-M45, W-M46, W-M47, W-M48, W-M49, W-M50, W-M51, W-M52, W-M53, W-M54, W-M55, W-M56, W-M57, W-M58, W-M59, W-M60, W-M61, W-M61, W-M62, W-M63, W-M64, W-M65, W-M66, W-M67, W-M68, W-M69, W-M70, W-M71, W-M72, W-M73, W-M74, W-M75, W-M76, W-M77, W-M79, W-M80, W-M81, W-M82, W-M83, W-M84, W-M85, W-M86, W-M87, W-M88, W-M89, W-M90, W-M91, W-M92, W-M93, W-M94, W-M95, W-M96, W-M97, W-M98, W-M99, W-M100,

wherein the biomarker is differentially present in samples of a subject with HCC and a subject with CLD.

Preferably, the protein is selected from

(A) I-M1, I-M3, I-M4, I-M5, I-M6, I-M7, I-M9, I-M11, I-M12, I-M13, I-M18, I-M19, I-M20, I-M21, I-M22, I-M23, I-M25, I-M26, I-M28, I-M32, I-M34, I-M36, I-M37, I-M41, I-M44, I-M46, I-M47, I-M52, I-M53, I-M64, I-M68, I-M69, I-M77, I-M79, I-M81, I-M84, I-M87, I-M88, I-M89, and I-M92

and/or

(B) W-M1, W-M2, W-M3, W-M4, W-M5, W-M7, W-M9, W-M10, W-M11, W-M12, W-M13, W-M14, W-M15, W-M16, W-M17, W-M18, W-M19, W-M20, W-M21, W-M22, W-M23, W-M25, W-M26, W-M27, W-M30, W-M31, W-M33, W-M34, W-M35, W-M36, W-M39, W-M40, W-M41, W-M43, W-M44, W-M46, W-M47, W-M48, W-M49, W-M50, W-M52, W-M53, W-M54, W-M55, W-M58, W-M60, W-M62, W-M63, W-M70, W-M71, W-M73, W-M76, W-M78, W-M84, W-M86, W-M88, W-M89, W-M90, W-M93, W-M95, W-M96, W-M98, and W-M100.

Biomarkers that, by themselves, are able to identify HCC include the I-M13, I-M18, I-M19, W-M2, and W-M23 protein biomarkers.

The present invention also provides a method for qualifying hepatocellular carcinoa risk in a patient, comprising (A) providing a spectrum generated by subjecting a biological sample from said patient to mass spectroscopic analysis that includes profiling on a chemically-derivatized affinity surface, and (B) putting the spectrum through pattern-recognition analysis that is keyed to at least one peak selected from the group consisting of

(i) I-M1, I-M3, I-M4, I-M5, I-M6, I-M7, I-M9, I-M11, I-M12, I-M13, I-M18, I-M19, I-M20, I-M21, I-M22, I-M23, I-M25, I-M26, I-M28, I-M32, I-M34, I-M36, I-M37, I-M41, I-M44, I-M46, I-M47, I-M52, I-M53, I-M64, I-M68, I-M69, I-M77, I-M79, I-M81, I-M84, I-M87, I-M88, I-M89, and I-M92

and/or the group consisting of

(ii) W-M1 , W-M2, W-M3, W-M4, W-M5, W-M7, W-M9, W-M10, W-M11, W-M12, W-M13, W-M14, W-M15, W-M16, W-M17, W-M18, W-M19, W-M20, W-M21, W-M22, W-M23, W-M25, W-M26, W-M27, W-M30, W-M31, W-M33, W-M34, W-M35, W-M36, W-M39, W-M40, W-M41, W-M43, W-M44, W-M46, W-M47, W-M48, W-M49, W-M50, W-M52, W-M53, W-M54, W-M55, W-M58, W-M60, W-M62, W-M63, W-M70, W-M71, W-M73, W-M76, W-M78, W-M84, W-M86, W-M88, W-M89, W-M90, W-M93, W-M95, W-M96, W-M98, and W-M100.

The pattern-recognition analysis may, for example, be keyed to a pair of peaks selected from the group consisting of

(A) I-M13 and I-M25, I-M13 and I-M7, I-M25 and I-M46, I-M37 and I-M77, I-M5 and I-M36

and/or the group consisting of

(B) W-M14 and W-M98, W-M21 and W-M46, W-M11 and W-M52, W-M16 and W-M89, W-M1 and W-M46, W-M21 and W-M76, W-M11 and W-M33, W-M13 and W-M18, W-M2 and W-M46, W-M33 and W-M54, W-M2 and W-M46, W-M16 and W-M46, W-M11 and W-M5.

Alternatively, the pattern-recognition analysis may be keyed to a triplet of peaks selected from the group consisting of

(A) I-M1, I-M4 and I-M36; I-M5, I-M7 and I-M19; I-M7, I-M19 and I-M46; I-M9, I-M34 and I-M52; I-M7, I-M18 and I-M47; I-M11, I-M13 and I-M36; I-M9, I-M77 and I-M84; and I-M18, I-M22 and I-M79

and/or the group consisting of

(B) W-M21, W-M22 and W-M35; W-M7, W-M21 and W-M46; W-M13, W-M14 and W-M98; W-M14, W-M54 and W-M70; W-M11, W-M33 and W-M46; W-M17, W-M36 and W-M98; W-M19, W-M21 and W-M22; W-M14, W-M15, W-M54; W-M55, W-M58 and W-M98; W-M11, W-M14 and W-M98; W-M1, W-M33 and W-M46; W-M40, W-M46 and W-M49; W-M15, W-M21 and W-M22; W-M14, W-M36 and W-M98; W-M5, W-M11 and W-M54; W-M14, W-M22 and W-M25; W-M14, W-M58 and W-M98; W-M5, W-M14 and W-M89; W-M7, W-M14 and W-M89; W-M14, W-M21 and W-M98; W-M11, W-M58 and W-M71; W-M14, W-M25 and W-M54; W-M14, W-M60 and W-M89; W-M21, W-M46 and W-M100.

In other embodiments, the pattern-recognition analysis may be keyed to a combination of more than three peaks, more particularly to a combination of 4, 5 or 6 peaks, where the combination is selected from the group consisting of

(A) I-M11, I-M13, I-M19 and I-M89; I-M13, I-M19, I-M22 and I-M26; I-M1, I-M5, I-M36 and I-M41; I-M19, I-M33, I-M44 and I-M46; I-M3, I-M18, I-M68 and I-M81; I-M3, I-M12, I-M34 and I-M81; I-M12, I-M13, I-M32 and I-M37; I-M18, I-M44, I-M46 and I-M79; I-M7, I-M13, I-M21 and I-M23; I-M3, I-M18, I-M77 and I-M92; I-M12, I-M13, I-M77 and I-M87; I-M6, I-M13, I-M34 and I-M81; I-M8, I-M19, I-M53, I-M64, I-M69; I-M4, I-M18, I-M28, I-M47 and I-M88; and I-M1, I-M4, I-M18, I-M36, I-M41 and I-M47

and/or the group consisting of

(B) W-M25, W-M55, W-M62 and W-M98; W-M7, W-M14, W-M17 and W-M89; W-M17, W-M31, W-M93 and W-M98; W-M11, W-M19, W-M46 and W-M50; W-M4, W-M33, W-M55 and W-M98; W-M5, W-M11, W-M36 and W-M54; W-M16, W-M36, W-M43 and W-M46; W-M11, W-M41, W-M54 and W-M73; W-M5, W-M11, W-M52 and W-M89; W-M4, W-M14, 58 and W-M89; W-M2, W-M12, W-M14, W-M89; W-M5, W-M11, W-M20 and W-M40; W-M21, W-M46, W-M70 and W-M88; W-M21, W-M33, W-M34 and W-M46; W-M17, W-M20, W-M40 and W-M58; W-M17, W-M33, W-M52 and W-M98; W-M3, W-M7, W-M21 and W-M46; W-M10, W-M22, W-M30 and W-M95; W-M1, W-M46, W-M54 and W-M70; W-M11, W-M14, W-M25 and W-M54; W-M11, W-M33, W-M46 and W-M90; W-M11, W-M14, W-M54 and W-M89; W-M7, W-M18, W-M21 and W-M22; W-M17, W-M20, W-M52 and W-M98; W-M2, W-M15, W-M19, W-M22 and W-M55; W-M17, W-M19, W-M26, W-M47 and W-M98; W-M9, W-M11, W-M27, W-M46 and W-M78; W-M5, W-M11, W-M33, W-M46 and W-M53; W-M2, W-M9, W-M15, W-M19 and W-M89; W-M5, W-M11, W-M52, W-M89 and W-M96; W-M16, W-M25, W-M40, W-M52 and W-M89; W-M14, W-M15, W-M21, W-M22 and W-M89; W-M5, W-M13, W-M16, W-M20 and W-M98; W-M9, W-M23, W-M26, W-M40 and W-M89; W-M20, W-M27, W-M30, W-M35, W-M40 and W-M70; W-M13, W-M26, W-M39, W-M44, W-M63 and W-M98; W-M5, W-M13, W-M35, W-M39, W-M86 and W-M89; and W-M3, W-M18, W-M21, W-M22, W-M48, and W-M84. In each case, the biomarker is differentially present in samples of a subject with HCC and a subject with CLD.

The invention also contemplates a kit for detecting and diagnosing HCC. Kits within the invention comprise, for example, (i) an adsorbent attached to a substrate that retains one or more of the biomarkers shown in FIG. 1 or FIG. 2, and (ii) instructions to detect the biomarker(s) by contacting a sample with the adsorbent and detecting the biomarker(s) retained by the adsorbent. An inventive kit may further comprise a washing solution and/or instructions for making a washing solution.

The present invention also provides software for qualifying hepatocellular carcinoma status in a subject, comprising an algorithm for analyzing data extracted from a spectrum generated by mass spectroscopic analysis of a biological sample taken from the subject, wherein said data relates to one or more biomarkers selected from either a first group consisting of

(i) I-M1, I-M2, I-M3, I-M4, I-M5, I-M6, I-M7, I-M8, I-M9, I-M10, I-M11, I-M12, I-M13, I-M14, I-M15, I-M16, I-M17, I-M18, I-M19, I-M20, I-M21, I-M22, I-M23, I-M24, I-M25, I-M26, I-M27, I-M28, I-M29, I-M30, I-M31, I-M32, I-M33, I-M34, I-M35, I-M36, I-M37, I-M38, I-M39, I-M40, I-M41, I-M42, I-M43, I-M44, I-M45, I-M46, I-M47, I-M48, I-M49, I-M50, I-M51, I-M52, I-M53, I-M54, I-M55, I-M56, I-M57, I-M58, I-M59, I-M60, I-M61, I-M61, I-M62, I-M63, I-M64, I-M65, I-M66, I-M67, I-M68, I-M69, I-M70, I-M71, I-M72, I-M73, I-M74, I-M75, I-M76, I-M77, I-M79, I-M80, I-M81, I-M82, I-M83, I-M84, I-M85, I-M86, I-M87, I-M88, I-M89, I-M90, I-M91, I-M92, I-M93, I-M94, I-M95, I-M96, I-M97, I-M98, I-M99, I-M100

or a second group consisting of

(ii) W-M1, W-M2, W-M3, W-M4, W-M5, W-M6, W-M7, W-M8, W-M9, W-M10, W-M11, W-M12, W-M13, W-M14, W-M15, W-M16, W-M17, W-M18, W-M19, W-M20, W-M21, W-M22, W-M23, W-M24, W-M25, W-M26, W-M27, W-M28, W-M29, W-M30, W-M31, W-M32, W-M33, W-M34, W-M35, W-M36, W-M37, W-M38, W-M39, W-M40, W-M41, W-M42, W-M43, W-M44, W-M45, W-M46, W-M47, W-M48, W-M49, W-M50, W-M51, W-M52, W-M53, W-M54, W-M55, W-M56, W-M57, W-M58, W-M59, W-M60, W-M61, W-M61, W-M62, W-M63, W-M64, W-M65, W-M66, W-M67, W-M68, W-M69, W-M70, W-M71, W-M72, W-M73, W-M74, W-M75, W-M76, W-M77, W-M79, W-M80, W-M81, W-M82, W-M83, W-M84, W-M85, W-M86, W-M87, W-M88, W-M89, W-M90, W-M91, W-M92, W-M93, W-M94, W-M95, W-M96, W-M97, W-M98, W-M99, W-M100,

The algorithm may carry out a pattern-recognition analysis that is keyed to data relating to at least one of the biomarkers. Alternatively, the algorithm may comprise classification tree analysis that is keyed to data relating to at least one of the biomarkers. In yet another embodiment, the algorithm comprises artificial neural network analysis that is keyed to data relating to at least one of the biomarkers

### BRIEF DESCRIPTION OF THE DRAWINGS

FIG. 1 is a list of the top 100 biomarkers identified with an IMAC3Cu ProteinChip® array format, ranked according to p value in a student t-test.

FIG. 2 is a list of the top 100 biomarkers identified with a WCX ProteinChip® array format, ranked according to p value in a student t-test.

### DETAILED DESCRIPTION OF THE PREFERRED EMBODIMENTS

In accordance with the present invention, a series of biomarkers associated with HCC has been discovered. In the present context, a biomarker is an organic biomolecule, particularly a polypeptide or protein, which is differentially present in a sample taken from a subject having HCC as compared to a comparable sample taken from a subject having CLD. A biomarker is present differentially in samples taken from HCC and CLD patients if it is present at an elevated level or a decreased level in samples of HCC patients as compared to samples of CLD patients that do not have HCC. More particularly, a biomarker is a polypeptide that is characterized by an apparent molecular weight, as determined by gas phase ion spectrometry, and that is present in samples from HCC subjects in an elevated or decreased level, as compared to CLD subjects. A biomarker is differentially present between two samples if the amount of the biomarker in one sample differs in a statistically significant way from the amount of biomarker in the other sample.

The biomarkers of the invention can be used to assess hepatocellular carcinoma status in a subject. “Hepatocellular carcinoma status” in this context subsumes, inter alia, the presence or absence of disease, the risk of developing disease, the stage of the disease, and the effectiveness of treatment of disease. Based on this status, further procedures may be indicated, including additional diagnostic tests or therapeutic procedures or regimens, such as endoscopy, biopsy, surgery, chemotherapy, immunotherapy, and radiation therapy. More particularly, the biomarkers of the invention are capable of identifying HCC and successfully distinguishing it from CLD. In some instances, a single biomarker is capable of identifying HCC with a predictive success of at least 85%, whereas, in other instances, a combination of biomarkers is used to obtain a predictive success of at least 85%. The biomarkers and combinations of biomarkers thus can be used to qualify HCC risk in a patient.

In some instances, a single biomarker is capable of identifying hepatocellular carcinoma with a sensitivity or specificity of at least 85%, whereas, in other instances, a combination or plurality of biomarkers is used to obtain a sensitivity or specificity of at least 85%. Thus, the biomarkers and combinations of biomarkers can be used to qualify hepatocellular carcinoma status in a subject or patient.

The biomarkers according to the invention are present in serum. The biological sample used according to the present invention, however, need not be a serum sample. Thus, a biological sample for qualifying hepatocellular carcinoma status may be a serum, plasma or blood sample, although serum samples are preferred.

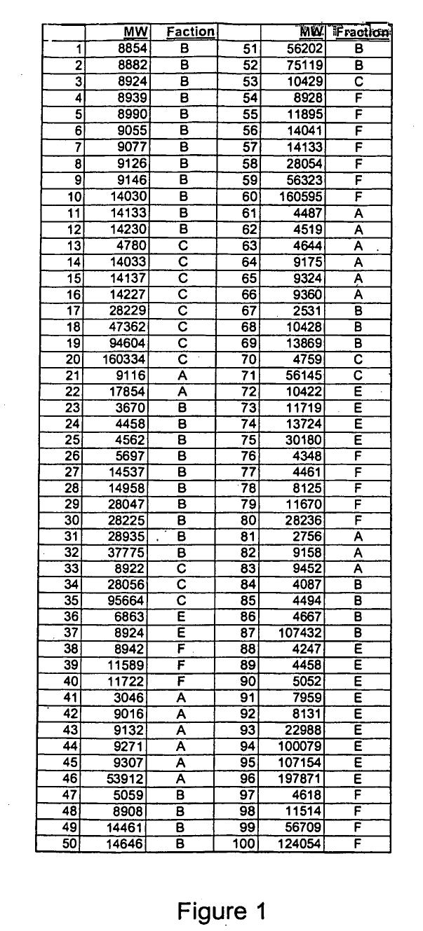

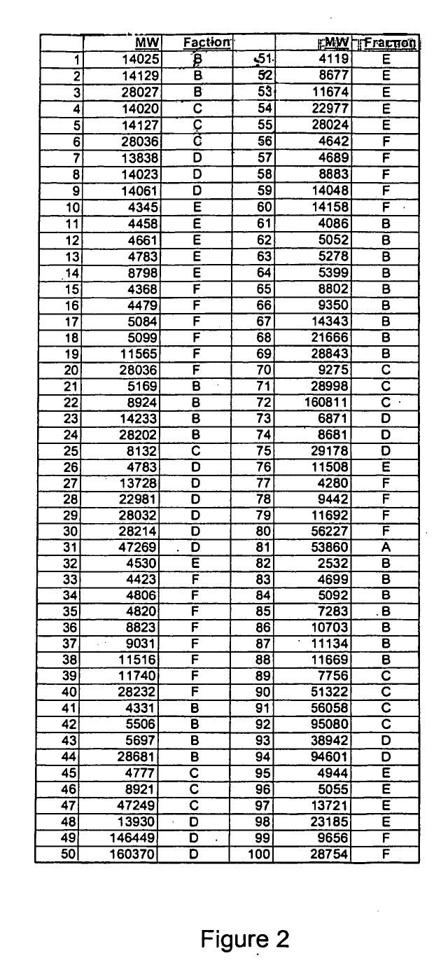

All of the biomarkers are characterized by molecular weight, and two lists of biomarkers within the present invention are provided in FIGS. 1 and 2. These figures list the top 100 biomarkers, as determined statistically by p value, that are identified by Cu(II)IMAC3 and WCX2 ProteinChip® array protocols described herein, respectively. In each figure, the number in the first column is the biomarker identifier. Thus, the first row in FIG. 1 relates to biomarker I-M1, the second row relates to biomarker I-M2, and so forth (“I-M” denoting biomarkers identified with the IMAC chip). Similarly, the first row in FIG. 2 relates to biomarker W-M1 and the second row relates to biomarker W-M2 (“W-M” denoting biomarkers identified with the WCX2 chip). The number in the second column of the figures is the apparent molecular weight of the biomarker in daltons, as determined by gas phase ion spectrometry. The letter in the final column of the figures denotes the fraction in which the biomarker elutes in the protocol described herein; that is, biomarkers with an “A” elute in first fraction, biomarkers with a “B” elute in the second fraction, and so forth. The fraction in which the biomarker elutes correlates with its pI, which biomarkers eluting at higher pH having a higher pI, and biomarkers eluting at lower pH having a lower pI.

Presenting the mass and affinity characteristics of a given biomarker within the invention, as in this description, characterizes that biomarker so as allow one to obtain and measured it, in accordance with the teachings herein. If desired, any of the biomarkers can be sequenced, in order to obtain an amino acid sequence, but this is not required to practice the present invention.

For example, a biomarker can be peptide mapped with a number of enzymes, such as trypsin and V8 protease, and the molecular weights of the digestion fragments can be used to search databases for sequences that match the molecular weights of the digestion fragments generated by the various enzymes. Alternatively, if the biomarkers are not proteins included in known databases, degenerate probes can be made based on the N-terminal amino acid sequence of the biomarker, which then are used to screen a genomic or cDNA library created from a sample from which the biomarker was initially detected. The positive clones can be identified, amplified, and their recombinant DNA sequences can be subcloned using techniques which are well known. Finally, protein biomarkers can be sequenced using protein ladder sequencing. Protein ladders can be generated by fragmenting the molecules and subjecting fragments to enzymatic digestion or other methods that sequentially remove a single amino acid from the end of the fragment. The ladder is then analyzed by mass spectrometry. The difference in masses of the ladder fragments identifies the amino acid removed from the end of the molecule.

The serum biomarkers according to the present invention were identified by comparing mass spectra of samples derived from sera from two groups of newly-diagnosed subjects, subjects with HCC and subjects with CLD. The subjects were diagnosed according to standard clinical criteria. HCC subjects were confirmed histologically, and CLD subjects were followed for at least 18 months following serum collection for any sign of HCC, to exclude subjects with asymptomatic HCC.

Sera from each group of subjects was collected, and fractionated with Q Ceramic HyperDF ion exchange resin (Biosepra, Cipergen Biosystems, Inc.) into six fractions which eluted at different pH. Fraction A comprised the flow through plus pH 9 eluant, Fraction B comprised the pH 7 eluant, Fraction C comprised the pH 5 eluant, Fraction D comprised the pH 4 eluant, Fraction E comprised the pH 3 eluant, and Fraction F comprised isopropyl alcohol/acetonitrile TFA eluant.

Each fraction was diluted and applied to a ProteinChip® array, either an Cu(II) (IMAC3) or WCX2 chip array. Both of these chip arrays are produced by Ciphergen Biosystems, Inc. (Fremont, Calif.).

The Cu(II) IMAC3 is an “immobilized metal affinity-capture” chip, with a nitrilotriacetic acid surface for high-capacity copper binding and subsequent affinity capture of proteins with metal binding residues. Imidazole may be used in binding and washing solutions to moderate protein binding, including binding of non-specific proteins. Increasing the concentration of imidazole in the washing buffers reduces the binding of the target proteins It is produced by photopolymerizing 5-methylacylamido-2-(N,N-biscarboxymethylamino)pentanoic acid (7.5 wt %) and N,N′-methylenebisacrylamide (0.4 wt %) using (-) riboflavin (0.02 wt %) as a photoinitiator. The monomer solution is deposited onto the chip substrate and irradiated to photopolymerize. The chip then is activated with Cu(II).

The WCX2 is a weak cation exchange array with a carboxylate surface to bind cationic proteins. The negatively charged carboxylate groups on the surface of the WCX2 chip interact with the positive charges exposed on the target proteins. The binding of the target proteins is reduced by increasing the concentration of salt or by increasing the pH of the washing buffers.

Following application of the eluant fraction, the chips were incubated to allow the polypeptides in the eluant to bind to the sites on the chip by an affinity interaction. After incubation, each chip array was washed to remove polypeptides that bind non-specifically and buffer contaminants. That chip then was dried, and an energy absorbing molecule or matrix was applied to it, to facilitate desorption and ionization in a mass spectrometer.

In the mass spectrometer, retained polypeptides were eluted from the chip array by laser desorption and ionization in a ProteinChip® Reader, which is integrated with ProteinChip® Software and a personal computer to analyze proteins captured on chip arrays. The ion optic and laser optic technologies in the ProteinChip® Reader detects proteins ranging from small peptides of less than 1000 Da up to proteins of 300 kilodaltons or more, and calculates the mass based on time-of-flight. Ionized polypeptides were detected and their mass accurately determined by this Time-of-Flight (TOF) Mass Spectrometry.

The mass spectra obtained for each group were subjected to scatter plot analysis, to eliminate run-to-run variation. Protein clusters on the scatter plot were eliminated, as potential biomarkers, that had the same pattern for both HCC and CLD, i.e, protein clusters that were either elevated for both conditions or depressed for both conditions. The remaining polypeptides were analyzed further for their ability to distinguish accurately between HCC and CLD. A student t-test analysis was employed to compare HCC and CLD groups for each protein cluster in the scatter plot, and protein clusters were selected that differed significantly (p<0.001) between the two groups.

Because the molecular weights were derived from scatter plot analysis, and because of limits on the ability of mass spectrometry to resolve molecular weights, the “absolute” molecular weight values given in FIGS. 1 and 2 actually represent approximate molecular weights. Thus, a given molecular weight for a biomarker should be interpreted as the midpoint of a molecular-weight range. The range surrounding the “absolute” value given in the figure is no more than ±0.15% (8840 to 8867 for I-M1), generally no more than ±0.10% (8844 to 8863 for I-M1), and often as small as ±0.05% (8850 to 8858 daltons for I-M1).

In an alternative embodiment, a process called “Significant Analysis of Microarray” (SAM) protein filtering was used to identify potential biomarkers. The protein filtering process was performed with SAM algorithms originally developed for cDNA/oligonucleotide microarray analysis. Tusher et al., “Significance analysis of microarrays applied to the ionizing radiation response,” Proc. Nat'l Acad. Sci. USA 98: 5116-21 (2001). Given the group identities, SAM was used to compare the normalized log₁₀ proteomic data between the tumor (40 HCC cases) and control (20 CLD cases with AFP <500 ng/mL) groups, and to identify the proteomic features which were significantly different at a median false significant value <0.000005. The control group was defined as “1” while the tumour group was defined as “2”. The “two classes unpaired data” was selected as the data-type. A total of 1000 times permutations were performed.

A total of 2384 proteomic features were found among the serum samples: 1087 by using the IMAC3 copper ProteinChip Array and 1297 by using the WCX2 ProteinChip Array. SAM for protein filtering was used to search for the serum proteins/polypeptides significantly different between the HCC and CLD cases. By setting the median value of false significant number <0.000005, 79 proteomic features were identified to be significantly higher in the HCC patient sera, and 160 proteomic features were significantly lower. Thus, 239 potential serological markers for the identification of HCC were found, in total. Table 1 lists five each of the most significantly higher and lower proteomic features.

TABLE 1
The five most significantly higher and the five most significantly
lower proteomic features for distinguishing between HCC and CLD

| | | Anion | Average intensity | |
|---|---|---|---|---|
| Proteomic | | exchange | of HCC cases | |
| feature | ProteinChip | fraction | (relative to CLD | |
| (M/Z value) | array used | number | cases) | p-value |
| 8944 | IMAC3 | 6 | 2 | 2 × 10⁻⁹ |
| I-M38 | copper | | | |
| 4568 | IMAC3 | 2 | 1.8 | 1 × 10⁻⁷ |
| I-M25 | copper | | | |
| 8930 | IMAC3 | 2 | 1.6 | 8 × 10⁻⁸ |
| I-M4 | copper | | | |
| 9117 | IMAC3 | 1 | 1.6 | 1 × 10⁻⁷ |
| I-M21 | copper | | | |
| 9327 | IMAC3 | 1 | 1.6 | 1 × 10⁻⁶ |
| I-M65 | copper | | | |
| 5175 | WCX2 | 2 | 0.7 | 2 × 10⁻⁶ |
| W-M26 | | | | |
| 14042 | IMAC3 | 2-6 | 0.6 | 1 × 10⁻⁷ |
| I-M56 | copper | | | |
| 14044 | WCX2 | 2-6 | 0.5 | 1 × 10⁻⁵ |
| W-M59 | | | | |
| 47434 | IMAC3 | 3 | 0.5 | 5 × 10⁻⁵ |
| I-M18 | copper | | | |
| 8811 | WCX2 | 5 | 0.4 | 2 × 10⁻⁵ |
| W-M14 | | | | |

Two approaches were used to determine whether a potential biomarker had predictive value in assessing HCC. By a first approach, Biomarker Pattern Software® (Ciphergen Biosystems, Fremont, Calif.) was employed to determine whether a potential biomarker has predictive value in assessing hepatocellular carcinoma. Biomarker Pattern Software® embodies a sophisticated, multivariate analysis program for identifying hidden correlations and patterns from SELDI protein profiles.

The second approach entailed artificial neural network (ANN) analysis. That is, an ANN model comprising the differential proteomic features was developed, to compute diagnostic scores for differentiation HCC from CLD. The ANN algorithm applies artificial intelligence to classification, pattern recognition, and prediction, as described, for example, by Poon et al., Oncology 61:275-83 (2001), and Xu et al., Cancer Res. 62:3493-7 (2002). An ANN model consists of processing elements (neurones), which are organised in layers. From a training data set, an ANN model can “learn” the association patterns between the input variables and outcomes, and then apply these patterns to new cases. The ANN model was developed with EasyNN (version 8.1, Stephen Wolstenholme, Cheshire, UK).

The development method was of the feed-forward type, and the networks were trained by weighted back-propagation. Both learning rate and momentum were optimised automatically by the software. The ANN model was composed of three layers, one input layer, one hidden layer and one output layer. There were seven nodes in the middle-hidden layer. The input variables for the development of the ANN model were the relative levels of the significant proteomic features whereas the output variable was the diagnostic score (range 0-1.0000) of each case. During training the ANN model, the diagnostic scores were defined as 0.0000 and 1.0000 for the CLD cases and HCC cases, respectively. With the developed ANN model, 10-fold cross-validation was performed to calculate the ANN diagnostic scores for each HCC and CLD cases. Cross-validation analysis showed that the sensitivity and specificity of the ANNs trained from the data set were 92.5% and 90%, respectively. Moreover, the ANNs correctly classified all the AFP-unidentified HCC cases with AFP levels below 500 ng/mL. In addition, one unseen CLD case with an AFP level of 903 ng/mL, and three unseen pooled serum samples from HCC cases with AFP >500 ng/mL, HCC cases with AFP <500 ng/mL, and from CLD cases, were all correctly classified by the ANNs. Similar results were obtained with biomarkers identified with the WCX2 chip. Receiver-operator characteristic (ROC) curves were constructed by calculating the sensitivities and specificities of tests at different cut-off points of the ANN diagnostic scores for differentiating HCC cases from CLD cases.

The diagnostic scores of the HCC cases (0.8985±0.2689) were significantly higher (p<0.0005, Mann-Whitney test) than those of CLD cases (0.1647±0.3091). The ROC curve analyses showed that ANN diagnostic score was useful in the differentiation between HCC and CLD cases regardless of serum AFP levels. The area under ROC curve was 0.934 (95% Cl: 0.871-0.996, p<0.0005) for all cases whereas the area was 0.966 (95% Cl: 0.917-1.015, p<0.0005) for cases with non-diagnostic serum AFP levels (<500 ng/mL). At an ANN diagnostic score cutoff of 0.5000, the sensitivity and specificity were 93% (37 out of 40 HCC cases, SE of 4%) and 90% (18 out of 20 control cases, SE of 7%), respectively. For HCC cases with non-diagnostic AFP levels, 95% of HCC cases (21 out of 22 cases, SE of 5%) were correctly classified. Alternatively, classification tree analysis was used to identify biomarkers and combinations of biomarkers with the highest predictive value. In this method, the sample data of the potential biomarkers was subjected to standard classification tree development using the S-plus (version 4.5), a statistical software package marketed by MathSoft, Inc. (Cambridge, Mass.).

In addition to analyzing the predictive value of proteomic features, additional information relating to proteomic features identified from SAM was obtained by two-way hierarchical clustering analysis. Before the analysis, the median intensity of each significant proteomic feature was normalized to equal to 1, and then all the normalized intensity data were subtracted by 1. After this data processing, the intensity data would be positive when it was greater than the median intensity, and negative when it was lower. The processed data of the significant proteomic features and the serum samples were subjected to two-way hierarchical clustering analysis, using the Cluster and TreeView, described by Eisen et al., Proc. Nat'l Acad. Sci. USA 95:14863-8 (1998). Pearson correlation (uncentered) was used to calculate the distance, and complete linkage clustering was performed.

Most of the typical CLD cases with AFP below 500 ng/mL (19 out of 20 cases), as well as one case with elevated serum AFP, were clustered together to form a distinctive group. The HCC cases were mainly clustered together. They formed one predominant subgroup, containing 17 cases, and several smaller subgroups.

In order to determine whether this HCC subgroup had elevated serum AFP levels, Mann-Whitney test was performed to compare the serum AFP levels between the cases of this subgroup and the rest of the HCC cases. The serum AFP levels of the predominant HCC subgroup were significantly higher (p=0.05). Therefore, the results demonstrate that, without knowing the serum AFP level, an HCC subtype with elevated AFP can be identified on the basis of the serum proteomic profiles. Thus, comprehensive serum proteomic profiling can classify HCC into different subtypes.

Of the 1087 protein clusters identified with the IMAC chip, student t-test analysis identified 137 of these as being statistically different (p<0.0001), whereas ANN analysis identified 151 protein clusters as potential biomarkers, identifying biomarkers that were not identified by t-test analysis. Some of these additional biomarkers were subsequently shown to have significant value in the detection of HCC.

Biomarkers and combinations of biomarkers identified in accordance with the present description may be used to qualify HCC risk in a patient. In particular, a biomarker or combination of biomarkers can be used to distinguish HCC patients from CLD patients with a high degree of predictive success, i.e., greater than at least 85%, preferably greater than at least 90%, and more preferably greater than 95%.

Biomarkers and combinations of biomarkers identified in accordance with the present description may be used to qualify hepatocellular carcinoma status in a subject. In particular, a biomarker or combination of biomarkers can be used to distinguish hepatocellular carcinoma patients from normal patients with a high degree of specificity or sensitivity, i.e., greater than at least 85%, preferably greater than at least 90%, and more preferably greater than 95%.

According to one aspect of the invention, therefore, the detection of biomarkers for diagnosis of hepatocellular carcinoma status entails contacting a sample from a subject with a substrate, e.g., a SELDI probe, having an adsorbent thereon, under conditions that allow binding between the biomarker and the adsorbent, and then detecting the biomarker bound to the adsorbent by gas phase ion spectrometry, for example, mass spectrometry. Other detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one aspect, the markers of this invention are detected by gas phase ion spectrometry, which involves the use of a gas phase ion spectrometer to detect gas phase ions. A gas phase ion spectrometer is an apparatus that detects gas phase ions. Gas phase ion spectrometers include an ion source that supplies gas phase ions. Gas phase ion spectrometers include, for example, mass spectrometers, ion mobility spectrometers, and total ion current measuring devices.

“Mass spectrometer” refers to a gas phase ion spectrometer that measures a parameter which can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions. “Laser desorption mass spectrometer” refers to a mass spectrometer which uses laser as a means to desorb, volatilize, and ionize an analyte.

“Mass analyzer” refers to a sub-assembly of a mass spectrometer that comprises means for measuring a parameter which can be translated into mass-to-charge ratios of gas phase ions. In a time-of flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the laser energy delivered per unit area of interrogated image. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons which ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

A preferred mass spectrometric technique for use in the invention is Surface Enhanced Laser Desorption and Ionization (SELDI), as described, for example, in U.S. Pat. No. 5,719,060 and No. 6,225,047, both to Hutchens and Yip, in which the surface of a probe that presents the analyte (here, one or more of the biomarkers) to the energy source plays an active role in desorption/ionization of analyte molecules. In this context, “probe” refers to a device adapted to engage a probe interface and to present an analyte to ionizing energy for ionization and introduction into a gas phase ion spectrometer, such as a mass spectrometer. A probe typically includes a solid substrate, either flexible or rigid, that has a sample-presenting surface, on which an analyte is presented to the source of ionizing energy.

One version of SELDI, called Surface-Enhanced Affinity Capture” or “SEAC,” involves the use of probes comprised of a chemically selective surface (“SELDI probe”). A “chemically selective surface” is one to which is bound either the adsorbent, also called a “binding moiety” or “capture reagent,” or a reactive moiety that is capable of binding a capture reagent, e.g., through a reaction forming a covalent or coordinate covalent bond.

The phrase “reactive moiety” here denotes a chemical moiety that is capable of binding a capture reagent. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. A “reactive surface” is a surface to which a reactive moiety is bound. An “adsorbent” or “capture reagent” can be any material capable of binding a biomarker of the invention. Suitable adsorbents for use in SELDI, according to the invention, are described in U.S. Pat. No. 6,225,047, supra.

One type of adsorbent is a “chromatographic adsorbent,” which is a material typically used in chromatography. Chromatographic adsorbents include, for example, ion exchange materials, metal chelators, immobilized metal chelates, hydrophobic interaction adsorbents, hydrophilic interaction adsorbents, dyes, simple biomolecules (e.g., nucleotides, amino acids, simple sugars and fatty acids), mixed mode adsorbents (e.g., hydrophobic attraction/electrostatic repulsion adsorbents). “Biospecific adsorbent” is another category, for adsorbents that contain a biomolecule, e.g., a nucleotide, a nucleic acid molecule, an amino acid, a polypeptide, a polysaccharide, a lipid, a steroid or a conjugate of these (e.g., a glycoprotein, a lipoprotein, a glycolipid). In certain instances the biospecific adsorbent can be a macromolecular structure such as a multiprotein complex, a biologicaI membrane or a virus. Illustrative biospecific adsorbents are antibodies, receptor proteins, and nucleic acids. A biospecific adsorbent typically has higher specificity for a target analyte than a chromatographic adsorbent.

Another version of SELDI is Surface-Enhanced Neat Desorption (SEND), which involves the use of probes comprising energy absorbing molecules that are chemically bound to the probe surface (“SEND probe”). The phrase “Energy absorbing molecules” (EAM) denotes molecules that are capable of absorbing energy from a laser desorption ionization source and, thereafter, contributing to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. The category also includes EAMs used in SELDI, as enumerated, for example, by U.S. Pat. No. 5,719,060 and U.S. Pat. No. 60/351,971 (Kitagawa), filed Jan. 25, 2002.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light. For instance, see U.S. Pat. No. 5,719,060. SEPAR and other forms of SELDI are readily adapted to detecting a biomarker or biomarker profile, pursuant to the present invention.

The detection of the biomarkers according to the invention can be enhanced by using certain selectivity conditions, e.g., adsorbents or washing solutions. The phrase “wash solution” refers to an agent, typically a solution, which is used to affect or modify adsorption of an analyte to an adsorbent surface and/or to remove unbound materials from the surface. The elution characteristics of a wash solution can depend, for example, on pH, ionic strength, hydrophobicity, degree of chaotropism, detergent strength, and temperature.

Pursuant to one aspect of the present invention, a sample is analyzed by means of a “biochip,” a term that denotes a solid substrate, having a generally planar surface, to which a capture reagent (adsorbent) is attached. Frequently, the surface of a biochip comprises a plurality of addressable locations, each of which has the capture reagent bound there. A biochip can be adapted to engage a probe interface and, hence, function as a probe in gas phase ion spectrometry preferably mass spectrometry. Alternatively, a biochip of the invention can be mounted onto another substrate to form a probe that can be inserted into the spectrometer.

A variety of biochips is available for the capture of biomarkers, in accordance with the present invention, from commercial sources such as Ciphergen Biosystems (Fremont, Calif.), Perkin Elmer (Packard BioScience Company (Meriden Conn.), Zyomyx (Hayward, Calif.), and Phylos (Lexington, Mass.). Exemplary of these biochips are those described in U.S. Pat. No. 6,225,047, supra, and U.S. Pat. No. 6,329,209 (Wagner et al.), and in PCT publications WO 99/51773 (Kuimelis and Wagner) and WO 00/56934 (Englert et al.).

More specifically, biochips produced by Ciphergen Biosystems have surfaces, presented on an aluminum substrate in strip form, to which are attached, at addressable locations, chromatographic or biospecific adsorbents. The surface of the strip is coated with silicon dioxide. Illustrative of Ciphergen ProteinChip® arrays are biochips H4, SAX-2, WCX-2, and IMAC-3, which include a functionalized, cross-linked polymer in the form of a hydrogel, physically attached to the surface of the biochip or covalently attached through a silane to the surface of the biochip. The H4 biochip has isopropyl functionalities for hydrophobic binding. The SAX-2 biochip has quaternary ammonium functionalities for anion exchange. The WCX-2 biochip has carboxylate functionalities for cation exchange. The IMAC-3 biochip has nitriloacetic acid functionalities that adsorb transition metal ions, such as Cu ++ and Ni++, by chelation. These immobilized metal ions, in turn, allow for adsorption of biomarkers by coordinate covalent bonding. Thus, Ciphergen's IMAC ProteinChip® arrays are sold with reactive moieties that become adsorbent upon the addition by the user of a metal solution.

In keeping with the above-described principles, a substrate with an adsorbent is contacted with the sample, containing serum, for a period of time sufficient to allow biomarker that may be present to bind to the adsorbent. In one embodiment of the invention, more than one type of substrate with adsorbent thereon is contacted with the biological sample. For example, a sample may be applied to both a WCX and an IMAC chip. This technique can allow for even more definitive assessment of cancer status. After the incubation period, the substrate is washed to remove unbound material. Any suitable washing solutions can be used; preferably, aqueous solutions are employed.

An energy absorbing molecule then is applied to the substrate with the bound biomarkers. As noted, an energy absorbing molecule is a molecule that absorbs energy from an energy source such as a laser, thereby assisting in desorption of biomarkers from the substrate. Exemplary energy absorbing molecules include, as noted above, cinnamic acid derivatives, sinapinic acid and dihydroxybenzoic acid. Preferably sinapinic acid is used.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Data generated by desorption and detection of biomarkers can be analyzed with the use of a programmable digital computer. The computer program analyzes the data to indicate the number of markers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set as zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample. Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates hepatocellular carcinoma status. Analysis of the data may be “keyed” to a variety of parameters that are obtained, either directly or indirectly, from the mass spectrometric analysis of the sample. These parameters include but are not limited to the presence or absence of one or more peaks, the shape of a peak or group of peaks, the height of one or more peaks, the log of the height of one or more peaks, and other arithmetic manipulations of peak height data.

In another aspect, the present invention provides kits for aiding in the diagnosis of hepatocellular carcinoma status, which kits are used to detect biomarkers according to the invention. The kits screen for the presence of biomarkers and combinations of biomarkers that are differentially present in samples from normal subjects and subjects with hepatocellular carcinoma.

In one embodiment, the kit comprises a substrate having an adsorbent thereon, wherein the adsorbent is suitable for binding a biomarker according to the invention, and a washing solution or instructions for making a washing solution, in which the combination of the adsorbent and the washing solution allows detection of the biomarker using gas phase ion spectrometry, e.g., mass spectrometry. The kit may include more than type of adsorbent, each present on a different substrate. In another embodiment, a kit of the invention may include a first substrate, comprising an adsorbent thereon, and a second substrate onto which the first substrate is positioned to form a probe, which can be inserted into a gas phase ion spectrometer, e.g., a mass spectrometer. In another embodiment, an inventive kit may comprise a single substrate that can be inserted into the spectrometer.

In a further embodiment, such a kit can comprise instructions for suitable operational parameters in the form of a label or separate insert. For example, the instructions may inform a consumer how to collect the sample or how to wash the probe. In yet another embodiment the kit can comprise one or more containers with biomarker samples, to be used as standard(s) for calibration.

In a preferred embodiment, the detection of biomarkers for diagnosis of hepatocellular carcinoma in a subject entails contacting a sample from a subject or patient, preferably a serum sample, with a substrate having an adsorbent thereon under conditions that allow binding between the biomarker and the adsorbent, and then detecting the biomarker bound to the adsorbent by gas phase ion spectrometry, preferably by Surface Enhanced Laser Desorption/Ionization (SELDI) mass spectrometry. The biomarkers are ionized by an ionization source such as a laser. The generated ions are collected by an ion optic assembly and accelerated toward an ion detector. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector-output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering. Thus, both the quantity and mass of the biomarker can be determined.

The detection of the biomarkers can be enhanced by using certain selectivity conditions, e.g., adsorbents or washing solutions. In one embodiment, the same or similar selectivity conditions that were used to discover the biomarkers are used in the method of detecting the biomarker in the sample. For example, immobilized metal affinity capture chips such as the Cu(II) IMAC3 and weak cationic exchange chips such as the WCX2 chips are preferred as the adsorbents for biomarker detection. However, other adsorbents can be used, as long as they have the binding characteristics suitable for binding the biomarkers.

More particularly, armed with the information regarding the biomarkers identified herein, one can use various methods to recognize patterns of doublets, triplets, and higher combinations of biomarkers according to the invention. These methods take raw data, regarding which peaks are present and their intensity, and provide a differential diagnosis of hepatocellular carcinoma versus normal for a sample.

Thus, a process of the invention can be divided into the learning phase and the classification phase. In the learning phase, a learning algorithm is applied to a data set that includes members of the different classes that are meant to be classified, for example, data from a plurality of samples diagnosed as cancer and data from a plurality of samples assigned a negative diagnosis. The methods used to analyze the data include, but are not limited to, artificial neural network, support vector machines, genetic algorithm and self-organizing maps and classification and regression tree analysis. These methods are described, for example, in WO 01/31579, May 3, 2001 (Barnhill eta/.); WO 02/06829, Jan. 24, 2002 (Hitt et al.) and WO 02/42733, May 30, 2002 (Paulse et al.). The learning algorithm produces a classifying algorithm. The classifier is keyed to elements of the data, such as particular markers and particular intensities of markers, usually in combination, that can classify an unknown sample into one of the two classes. The classifier is ultimately used for diagnostic testing.

Software, both freeware and proprietary software, is readily available to analyze such patterns in data, and to devise additional patterns with any predetermined criteria for success. Those biomarkers which by themselves are predictive of a differential diagnosis of hepatocellular carcinoma versus CLD do not require pattern recognition software to analyze the data.

The following examples are offered by way of illustration, and are not limiting.

### EXAMPLE I

### Patient Population

With the patients' consent, clotted blood samples were collected from 40 patients with HCC and 21 patients with chronic liver diseases at presentation, and stored at −70° C. before assay. Patients with HCC were diagnosed according to standard clinical criteria. All HCC cases were histologically confirmed. Among the HCC cases, 18 had serum AFP levels >500 ng/ml, and 22 had a serum AFP level <500 ng/mL. Serum samples from 20 patients with CLD and AFP <500 ng/ml were used as a control group. All CLD patients were followed for at least 18 months for any sign of HCC so as to exclude subjects with asymptomatic HCC. One serum sample from a CLD patient with AFP level >500 ng/mL (905 ng/mL) was also analyzed in this study. Aside from analysing each serum sample individually, serum samples from HCC patients with AFP >500 ng/ml, and those from HCC patients with AFP <500 ng/ml were pooled as samples HCCP1 and HCCP2 respectively, while serum samples from the control group were pooled as sample CLDP1. The serum AFP levels were measured by microparticle EIA (MEIA, Abbott Laboratories, Chicago, USA).

### EXAMPLE 2

### Fractionation of serum

Buffers:

1. U9 (9M urea, 2% CHAPS, 50 mM Tris-HCl pH9)

2. U1 (1M urea, 0.22% CHAPS, 50 mM Tris-HCl pH9)

3. wash buffer 1: 50 mM Tris-HCl with 0.1% n-octyl β-D-Glucopyranoside (OGP) pH9

4. wash buffer 2: 100 mM sodium phosphate with 0.1% OGP pH7

5. wash buffer 3: 100 mM sodium acetate with 0.1% OGP pH5

6. wash buffer 4: 100 mM sodium acetate with 0.1% OGP pH4

7. wash buffer 5: 50 mM sodium citrate with 0.1% OGP pH3

8. wash buffer 6: 33.3% isopropanol/16.7% acetonitrile/0.1% trifluoroacetic acid in water

Anion exchange fractionation can be regarded as analogous to the first dimensional separation, isoelectric focusing, in the 2D PAGE technology. Both technologies separate proteins on the basis of their pI values. Thirty microliters of U9 buffer were added to 20 μL of serum in a tube and were mixed at 4° C. for 20 minutes. Ion exchange resin (Q Ceramic HyperDF ion exchange resin, Biosepra SA, France) was washed 3 times with 5 bed volumes of 50 mM Tris-HCl pH9 and stored in 50% suspension. To each well of a 96-well filter plate (96-well Silent Screen filter plate, Loprodyne membrane, 0.45 micron pore, Nalge Nunc International, USA), 125 μL of ion exchange resin (50% suspension) was added on a Biomek 2000 Automation Workstation (Beckman Coulter, Fullerton, Calif.), washed 3 times with 150 μL U1 buffer, and vacuum dried. Urea-treated serum was transferred to each well of ion exchange resin. The serum tube was rinsed with 50 μL of U1 buffer, which was also transferred to the corresponding well in filter plate. The filter plate was mixed on a platform shaker at 4° C. for 30 minutes. Flow-through fraction was collected in a 96-well plate by vacuum suction (Fraction 1). Then, 100 μL of wash buffer 1 was added to each well of filter plate and mixed for 10 minutes at room temperature. Eluant was collected into the same 96-well plate (Fraction 1). Resins in the filter plate were subsequently washed two times each with 100 μL wash buffers 2, 3, 4, 5 and 6. Each eluant (total volume of 200 μL) was collected in a 96-well plate (Fractions 2, 3, 4, 5 and 6).

### EXAMPLE 3

### SELDI Analysis of Fractionated Serum

ProteinChip® Arrays were set up in 96-well bioprocessors. Buffer delivery and sample incubation were performed on a Biomek 2000 Automation Workstation. Each serum fraction was analyzed on IMAC3 (loaded with copper) and WCX2 ProteinChip® Arrays in duplicates. The different ProteinChip surfaces (2^{nd} dimension) helped to identify very low abundance proteins. The IMAC3 copper and WCX2 ProteinChip surfaces preferentially retain different groups of proteins according to their physiochemical properties.

The IMAC3 copper and WCX2 arrays (Ciphergen Biosystems Inc, Fremont, Calif.) were equilibrated two times with 150 μL of binding buffer (100 mM sodium phosphate+0.5M NaCl pH7 for IMAC3, 100 mM sodium acetate pH4 for WCX2). Each serum fraction was diluted in the corresponding binding buffer (⅕ dilution for IMAC3 and 1/10 dilution for WCX2) and 100 μL was applied to each ProteinChip® array. Incubation was performed on a platform shaker at room temperature for 30 minutes. Each array was washed three times with 150 μL of corresponding binding buffer and rinsed two times with water. ProteinChip® arrays were air-dried. Sinapinic acid matrix (prepared in 50% acetonitrile, 0.5% trifluoroacetic acid) was applied to each array.

ProteinChip® arrays were read on a ProteinChip®D PBSII Reader (Ciphergen Biosystems Inc.) to measure the masses and intensities of the protein peaks (Ciphergen). A total of 253 laser shots were averaged for each array. The mass spectrometric analysis (3^{rd} dimension) with the ProteinChip PBS II reader can be regarded as a higher resolution substitution of the 2^{nd} dimensional separation, SDS-PAGE, in the 2D PAGE technology. Both technologies separate the proteins on the basis of their molecular weights. 235 laser shots were averaged for each array with mass ranging from Oto 200 kDa. All the mass spectra were normalized to have the same total ion current. The CVs of the peak intensities were less than 15% (manufacturer information). Common protein peaks were picked by the Biomarker Wizard™ function of the ProteinChip Software (Ciphergen).

What is claimed is:

1. A method for qualifying hepatocellular carcinoma status in a subject, comprised of analyzing a biological sample from said subject for a diagnostic level of a protein selected from a first group consisting of
(A) I-M1, I-M2, I-M3, I-M4, I-M5, I-M6, I-M7, I-M8, I-M9, I-M10, I-M11, I-M12, I-M13, I-M14, I-M15, I-M16, I-M17, I-M18, I-M19, I-M20, I-M21, I-M22, I-M23, I-M24, I-M25, I-M26, I-M27, I-M28, I-M29, I-M30, I-M31, I-M32, I-M33, I-M34, I-M35, I-M36, I-M37, I-M38, I-M39, I-M40, I-M41, I-M42, I-M43, I-M44, I-M45, I-M46, I-M47, I-M48, I-M49, I-M50, I-M51, I-M52, I-M53, I-M54, I-M55, I-M56, I-M57, I-M58, I-M59, I-M60, I-M61, I-M61, I-M62, I-M63, I-M64, I-M65, I-M66, I-M67, I-M68, I-M69, I-M70, I-M71, I-M72, I-M73, I-M74, I-M75, I-M76, I-M77, I-M79, I-M80, I-M81, I-M82, I-M83, I-M84, I-M85, I-M86, I-M87, I-M88, I-M89, I-M90, I-M91, I-M92, I-M93, I-M94, I-M95, I-M96, I-M97, I-M98, I-M99, I-M100
and/or a second group consisting of
(B) W-M1, W-M2, W-M3, W-M4, W-M5, W-M6, W-M7, W-M8, W-M9, W-M10, W-M11, W-M12, W-M13, W-M14, W-M15, W-M16, W-M17, W-M18, W-M19, W-M20, W-M21, W-M22, W-M23, W-M24, W-M25, W-M26, W-M27, W-M28, W-M29, W-M30, W-M31, W-M32, W-M33, W-M34, W-M35, W-M36, W-M37, W-M38, W-M39, W-M40, W-M41, W-M42, W-M43, W-M44, W-M45, W-M46, W-M47, W-M48, W-M49, W-M50, W-M51, W-M52, W-M53, W-M54, W-M55, W-M56, W-M57, W-M58, W-M59, W-M60, W-M61, W-M61, W-M62, W-M63, W-M64, W-M65, W-M66, W-M67, W-M68, W-M69, W-M70, W-M71, W-M72, W-M73, W-M74, W-M75, W-M76, W-M77, W-M79, W-M80, W-M81, W-M82, W-M83, W-M84, W-M85, W-M86, W-M87, W-M88, W-M89, W-M90, W-M91, W-M92, W-M93, W-M94, W-M95, W-M96, W-M97, W-M98, W-M99, W-M100,
wherein said level is elevated relative to a norm.

2. A method for qualifying hepatocellular carcinoma status in a patient according to claim 1, wherein said protein is selected from the group consisting of
(A) I-M1, I-M3, I-M4, I-M5, I-M6, I-M7, I-M9, I-M11, I-M12, I-M13, I-M18, I-M19, I-M20, I-M21, I-M22, I-M23, I-M25, I-M26, I-M28, I-M32, I-M34, I-M36, I-M37, I-M41, I-M44, I-M46, I-M47, I-M52, I-M53, I-M64, I-M68, I-M69, I-M77, I-M79, I-M81, I-M84, I-M87, I-M88, I-M89, and I-M92
and/or a second group consisting of
(B) W-M1, W-M2, W-M3, W-M4, W-M5, W-M7, W-M9, W-M10, W-M11, W-M12, W-M13, W-M14, W-M15, W-M16, W-M17, W-M18, W-M19, W-M20, W-M21, W-M22, W-M23, W-M25, W-M26, W-M27, W-M30, W-M31, W-M33, W-M34, W-M35, W-M36, W-M39, W-M40, W-M41, W-M43, W-M44, W-M46, W-M47, W-M48, W-M49, W-M50, W-M52, W-M53, W-M54, W-M55, W-M58, W-M60, W-M62, W-M63, W-M70, W-M71, W-M73, W-M76, W-M78, W-M84, W-M86, W-M88, W-M89, W-M90, W-M93, W-M95, W-M96, W-M98, and W-M100.

3. A method according to claim 2, wherein said protein is I-M13, I-M18, I-M19, W-M2, or W-M23.

4. A method for qualifying hepatocellular carcinoma risk in a patient, comprising
(A) providing a spectrum generated by mass spectroscopic analysis of a biological sample taken from the subject, and
(B) extracting data from the spectrum and subjecting the data to pattern-recognition analysis that is keyed to at least one peak selected from
a first group consisting of
(i) I-M1, I-M3, I-M4, I-M5, I-M6, I-M7, I-M9, I-M11, I-M12, I-M13, I-M18, I-M19, I-M20, I-M21, I-M22, I-M23, I-M25, I-M26, I-M28, I-M32, I-M34, I-M36, I-M37, I-M41, I-M44, I-M46, I-M47, I-M52, I-M53, I-M64, I-M68, I-M69, I-M77, I-M79, I-M81, I-M84, I-M87, I-M88, I-M89, and I-M92,
and/or a second group consisting of
(ii) W-M1, W-M2, W-M3, W-M4, W-M5, W-M7, W-M9, W-M10, W-M11, W-M12, W-M13, W-M14, W-M15, W-M16, W-M17, W-M18, W-M19, W-M20, W-M21, W-M22, W-M23, W-M25, W-M26, W-M27, W-M30, W-M31, W-M33, W-M34, W-M35, W-M36, W-M39, W-M40, W-M41, W-M43, W-M44, W-M46, W-M47, W-M48, W-M49, W-M50, W-M52, W-M53, W-M54, W-M55, W-M58, W-M60, W-M62, W-M63, W-M70, W-M71, W-M73, W-M76, W-M78, W-M84, W-M86, W-M88, W-M89, W-M90, W-M93, W-M95, W-M96, W-M98, and W-M100.

5. A method according to claim 4, wherein said pattern-recognition analysis is keyed to a pair of peaks selected from
(A) I-M13 and I-M25, I-M13 and I-M7, I-M25 and I-M46, I-M37 and I-M77, I-M5 and I-M36, and/or
(B) W-M14 and W-M98, W-M21 and W-M46, W-M11 and W-M52, W-M16 and W-M89, W-M1 and W-M46, W-M21 and W-M76, W-M11 and W-M33, W-M13 and W-M18, W-M2 and W-M46, W-M33 and W-M54, W-M2 and W-M46, W-M16 and W-M46, W-M11 and W-M5.

6. A method according to claim 4, wherein said pattern-recognition analysis is keyed to a triplet of peaks selected from
(A) I-M1, I-M4 and I-M36; I-M5, I-M7 and I-M19; I-M7, I-M19 and I-M46; I-M9, I-M34 and I-M52; I-M7, I-M18 and I-M47; I-M11, I-M13 and I-M36; I-M9, I-M77 and I-M84; and [-M18, I-M22 and I-M79, and/or
(B) W-M21, W-M22 and W-M35; W-M7, W-M21 and W-M46; W-M13, W-M14 and W-M98; W-M14, W-M54 and W-M70; W-M11, W-M33 and W-M46; W-M17, W-M36 and W-M98; W-M19, W-M21 and W-M22; W-M14, W-M15, W-M54; W-M55, W-M58 and W-M98; W-M11, W-M14 and W-M98; W-M1, W-M33 and W-M46; W-M40, W-M46 and W-M49; W-M15, W-M21 and W-M22; W-M14, W-M36 and W-M98; W-M5, W-M11 and W-M54; W-M14, W-M22 and W-M25; W-M14, W-M58 and W-M98; W-M5, W-M14 and W-M89; W-M7, W-M14 and W-M89; W-M14, W-M21 and W-M98; W-M11, W-M58 and W-M71; W-M14, W-M25 and W-M54; W-M14, W-M60 and W-M89; W-M21, W-M46 and W-M100.

7. A method according to claim 4, wherein said pattern-recognition analysis is keyed to a combination of peaks selected from
(A) I-M11, I-M13, I-M19 and I-M89; I-M13, I-M19, I-M22 and I-M26; I-M1, I-M5, I-M36 and I-M41; I-M19, I-M33, I-M44 and I-M46; I-M3, I-M18, I-M68 and I-M81; I-M3, I-M12, I-M34 and I-M81; I-M12, I-M13, I-M32 and I-M37; I-M18, I-M44, I-M46 and I-M79; I-M7, I-M13, I-M21 and I-M23; I-M3, I-M18, I-M77 and I-M92; I-M12, I-M13, I-M77 and I-M87; I-M6, I-M13, I-M34 and I-M81; I-M8, I-M19, I-M53, I-M64, I-M69; I-M4, I-M18, I-M28, I-M47 and I-M88; and I-M1, I-M4, I-M18, I-M36, I-M41 and I-M47, and/or
(B) W-M25, W-M55, W-M62 and W-M98; W-M7, W-M14, W-M17 and W-M89; W-M17, W-M31, W-M93 and W-M98; W-M11, W-M19, W-M46 and W-M50; W-M4, W-M33, W-M55 and W-M98; W-M5, W-M11, W-M36 and W-M54; W-M16, W-M36, W-M43 and W-M46; W-M11, W-M41, W-M54 and W-M73; W-M5, W-M11, W-M52 and W-M89; W-M4, W-M14, 58 and W-M89; W-M2, W-M12, W-M14, W-M89; W-M5, W-M11, W-M20 and W-M40; W-M21, W-M46, W-M70 and W-M88; W-M21, W-M33, W-M34 and W-M46; W-M17, W-M20, W-M40 and W-M58; W-M17, W-M33, W-M52 and W-M98; W-M3, W-M7, W-M21 and W-M46; W-M10, W-M22, W-M30 and W-M95; W-M1, W-M46, W-M54 and W-M70; W-M11, W-M14, W-M25 and W-M54; W-M11, W-M33, W-M46 and W-M90; W-M11, W-M14, W-M54 and W-M89; W-M7, W-M18, W-M21 and W-M22; W-M17, W-M20, W-M52 and W-M98; W-M2, W-M15, W-M19, W-M22 and W-M55; W-M17, W-M19, W-M26, W-M47 and W-M98; W-M9, W-M11, W-M27, W-M46 and W-M78; W-M5, W-M11, W-M33, W-M46 and W-M53; W-M2, W-M9, W-M15, W-M19 and W-M89; W-M5, W-M11, W-M52, W-M89 and W-M96; W-M16, W-M25, W-M40, W-M52 and W-M89; W-M14, W-M15, W-M21, W-M22 and W-M89; W-M5, W-M13, W-M16, W-M20 and W-M98; W-M9, W-M23, W-M26, W-M40 and W-M89; W-M20, W-M27, W-M30, W-M35, W-M40 and W-M70; W-M13, W-M26, W-M39, W-M44, W-M63 and W-M98; W-M5, W-M13, W-M35, W-M39, W-M86 and W-M89; and W-M3, W-M18, W-M21, W-M22, W-M48, and W-M84.

8. A kit for detecting and diagnosing hepatocelluar carcinoma, comprising
(A) an adsorbent attached to a substrate that retains one or more of the biomarkers selected from either a first group consisting of
(i) I-M1, I-M2, I-M3, I-M4, I-M5, I-M6, I-M7, I-M8, I-M9, I-M10, I-M11, I-M12, I-M13, I-M14, I-M15, I-M16, I-M17, I-M18, I-M19, I-M20, I-M21, I-M22, I-M23, I-M24, I-M25, I-M26, I-M27, I-M28, I-M29, I-M30, I-M31, I-M32, I-M33, I-M34, I-M35, I-M36, I-M37, I-M38, I-M39, I-M40, I-M41, I-M42, I-M43, I-M44, I-M45, I-M46, I-M47, I-M48, I-M49, I-M50, I-M51, I-M52, I-M53, I-M54, I-M55, I-M56, I-M57, I-M58, I-M59, I-M60, I-M61, I-M61, I-M62, I-M63, I-M64, I-M65, I-M66, I-M67, I-M68, I-M69, I-M70, I-M71, I-M72, I-M73, I-M74, I-M75, I-M76, I-M77, I-M79, I-M80, I-M81, I-M82, I-M83, I-M84, I-M85, I-M86, I-M87, I-M88, I-M89, I-M90, I-M91, I-M92, I-M93, I-M94, I-M95, I-M96, I-M97, I-M98, I-M99, I-M100
or a second group consisting of
(ii) W-M1, W-M2, W-M3, W-M4, W-M5, W-M6, W-M7, W-M8, W-M9, W-M10, W-M11, W-M12, W-M13, W-M14, W-M15, W-M 16, W-M17, W-M18, W-M19, W-M20, W-M21, W-M22, W-M23, W-M24, W-M25, W-M26, W-M27, W-M28, W-M29, W-M30, W-M31, W-M32, W-M33, W-M34, W-M35, W-M36, W-M37, W-M38, W-M39, W-M40, W-M41, W-M42, W-M43, W-M44, W-M45, W-M46, W-M47, W-M48, W-M49, W-M50, W-M51, W-M52, W-M53, W-M54, W-M55, W-M56, W-M57, W-M58, W-M59, W-M60, W-M61, W-M61, W-M62, W-M63, W-M64, W-M65, W-M66, W-M67, W-M68, W-M69, W-M70, W-M71, W-M72, W-M73, W-M74, W-M75, W-M76, W-M77, W-M79, W-M80, W-M81, W-M82, W-M83, W-M84, W-M85, W-M86, W-M87, W-M88, W-M89, W-M90, W-M91, W-M92, W-M93, W-M94, W-M95, W-M96, W-M97, W-M98, W-M99, W-M100,
(B) instructions to detect the biomarker(s) by contacting a sample with the adsorbent and detecting the biomarker(s) retained by the adsorbent.

9. A kit according to claim 8, further comprising a washing solution or instructions for making a washing solution.

10. A kit according to claim 8, wherein the substrate is a SELDI probe that comprises either (i) functionalities that adsorb transition metal ions by chelation or (ii) functionalities that allow for cation exchange.

11. Software for qualifying hepatocellular carcinoma status in a subject, comprising an algorithm for analyzing data extracted from a spectrum generated by mass spectroscopic analysis of a biological sample taken from the subject, wherein said data relates to one or more biomarkers selected from either a first group consisting of
(i) I-M1, I-M2, I-M3, I-M4, I-M5, I-M6, I-M7, I-M8, I-M9, I-M10, I-M11, I-M12, I-M13, I-M14, I-M15, I-M16, I-M17, I-M18, I-M19, I-M20, I-M21, I-M22, I-M23, I-M24, I-M25, I-M26, I-M27, I-M28, I-M29, I-M30, I-M31, I-M32, I-M33, I-M34, I-M35, I-M36, I-M37, I-M38, I-M39, I-M40, I-M41, I-M42, I-M43, I-M44, I-M45, I-M46, I-M47, I-M48, I-M49, I-M50, I-M51, I-M52, I-M53, I-M54, I-M55, I-M56, I-M57, I-M58, I-M59, I-M60, I-M61, I-M61, I-M62, I-M63, I-M64, I-M65, I-M66, I-M67, I-M68, I-M69, I-M70, I-M71, I-M72, I-M73, I-M74, I-M75, I-M76, I-M77, I-M79, I-M80, I-M81, I-M82, I-M83, I-M84, I-M85, I-M86, I-M87, I-M88, I-M89, I-M90, I-M91, I-M92, I-M93, I-M94, I-M95, I-M96, I-M97, I-M98, I-M99, I-M100
or a second group consisting of
(ii) W-M1, W-M2, W-M3, W-M4, W-M5, W-M6, W-M7, W-M8, W-M9, W-M10, W-M11, W-M12, W-M13, W-M14, W-M15, W-M16, W-M17, W-M18, W-M19, W-M20, W-M21, W-M22, W-M23, W-M24, W-M25, W-M26, W-M27, W-M28, W-M29, W-M30, W-M31, W-M32, W-M33, W-M34, W-M35, W-M36, W-M37, W-M38, W-M39, W-M40, W-M41, W-M42, W-M43, W-M44, W-M45, W-M46, W-M47, W-M48, W-M49, W-M50, W-M51, W-M52, W-M53, W-M54, W-M55, W-M56, W-M57, W-M58, W-M59, W-M60, W-M61, W-M61, W-M62, W-M63, W-M64, W-M65, W-M66, W-M67, W-M68, W-M69, W-M70, W-M71, W-M72, W-M73, W-M74, W-M75, W-M76, W-M77, W-M79, W-M80, W-M81, W-M82, W-M83, W-M84, W-M85, W-M86, W-M87, W-M88, W-M89, W-M90, W-M91, W-M92, W-M93, W-M94, W-M95, W-M96, W-M97, W-M98, W-M99, W-M100,

12. Software according to claim 11, wherein said algorithm carries out a pattern-recognition analysis that is keyed to data relating to at least one of the biomarkers.

13. Software according to claim 12, wherein said algorithm comprises classification tree analysis that is keyed to data relating to at least one of the biomarkers.

14. Software according to claim 12, wherein said algorithm comprises artificial neural network analysis that is keyed to data relating to at least one of the biomarkers
